(19) United States
(12) Patent Application Publication
(10) Pub. No.: US 2023/0093674 A1
(43) Pub. Date: Mar. 23, 2023

(54) PORTABLE INFUSION PUMP WITH NEGATIVE PRESSURE CONTROL

(71) Applicant: MODULAR MEDICAL, INC., San Diego, CA (US)

(72) Inventors: Paul M. DiPerna, Escondido, CA (US); Marc D. Goldman, San Diego, CA (US); David Neese, Escondido, CA (US); Patrick Hearty, Vista, CA (US)

(21) Appl. No.: 17/482,848

(22) Filed: Sep. 23, 2021

(51) Int. Cl. A61M 5/142; A61M 5/168; A61M 5/148

(52) U.S. Cl. CPC A61M 5/14244; A61M 5/16854; A61M 5/14216; A61M 5/16813; A61M 5/1483; A61M 2205/3331

(57) ABSTRACT

A portable system and method for infusing fluid medicaments to a patient through an elastomeric fluid channel includes an airtight pressure shell for holding a collapsible bag of the fluid medicament to be infused. A pressure sensor connected to the pressure shell monitors decreases below ambient pressure inside the shell to determine when the ambient pressure becomes insufficient to continue assisting the withdrawal of fluid from the collapsible bag, to an external pinch/squeeze unit. At this point, an equilibrator operates to reestablish ambient pressure in the pressure shell for continued operation. In operation, the pinch/squeeze unit exerts radial forces on the elastomeric fluid channel. These forces act to sequentially push fluid medicament for infusion to the patient when the elastomeric fluid channel is occluded and stressed, and draw fluid medicament from the collapsible bag as the elastomeric fluid channel is unstressed.

### FIELD OF THE INVENTION

The present invention pertains to systems and methods for infusing fluid medicaments from a collapsible bag to a patient. More particularly, the present invention pertains to portable pumps that establish and create infusion pressures on the collapsible bag which are below ambient pressure. The present invention is particularly, but not exclusively, useful as an infusion system wherein motive forces interact radially with an elastomeric fluid channel carrying the fluid medicament. Wherein this interaction is controlled by monitoring decreases below ambient air pressure acting on a collapsible bag containing the fluid medicament, and equilibrating the pressure shell to ambient pressure when an overpressure is insufficient to infuse fluid medicament to the patient in accordance with a predetermined protocol.

### BACKGROUND OF THE INVENTION

Insulin infusion pumps typically have several characteristics in common. Namely, they are preferably light-weight, portable, conveniently operable, comfortable and, most importantly, they are operationally accurate and reliable. To achieve these goals, many different methods have been employed for infusing a fluid medicament to a patient. In each case, it is essential that the fluid medicament be somehow accurately and reliably moved from the source of fluid medicament to the patient.

Typically, many infusion pumps function by generating a mechanical pressure on fluid medicament at its source. Also, using a different functionality, peristaltic pumps operate by direct engagement with an elongated elastomeric fluid channel, and imposing axially directed forces against the fluid as it moves through the infusion tube. Various combinations of these functionalities are also possible.

Apart from the traditional methods for moving a fluid through a tube, the present invention recognizes that the reactionary forces acting within a resilient elastomeric fluid channel as it relaxes and transitions from a stressed configuration back to an unstressed configuration can also be beneficially employed to assist fluid flow. Further, the present invention also recognizes that properly employed sub-ambient pressures on fluid medicament in a collapsible bag can also be beneficial for this same purpose. Moreover, the present invention recognizes that the combined efforts of elastomeric reflex and sub-ambient over-pressures can allow for an effective pumping action for an insulin infusion pump.

With the above in mind, it is an object of the present invention to provide a portable infusion pump that provides sub-ambient over-pressures on a collapsible fluid medicament bag for its operation without relying on a mechanical pump. Another object of the present invention is to provide a portable infusion pump which employs the radial effect of elastomeric reflex from an infusion tube as a primary means for its pumping function. Still another object of the present invention is to provide an infusion pump that, in combination, relies on sub-ambient pressures and elastomeric reflex forces for its pumping function. Yet another object of the present invention is to provide a portable infusion pump that is easy to use, simple to manufacture and relatively cost effective.

### SUMMARY OF THE INVENTION

In accordance with the present invention a portable pump is provided for infusing a fluid medicament to a patient from a replaceable infusion unit. As envisioned for the present invention, the infusion unit will include a collapsible bag which holds the fluid medicament, a cannula needle set which establishes fluid communication with the patient, and an elastomeric fluid channel which interconnects the collapsible bag in fluid communication with the cannula needle set.

Structurally, the portable pump requires a pressure shell which is adapted to create an airtight pressure chamber. The pressure chamber will have a volume V_{c} which is sufficient for holding the collapsible bag, while the elastomeric fluid channel extends from the collapsible bag, and further therefrom outside the pressure shell. A pressure sensor is mounted on the pressure shell to monitor chamber pressure, P_{c}, within the pressure chamber, and an equilibration valve is mounted on the pressure shell to periodically equilibrate the chamber pressure P_{c} with the ambient air pressure P_{amb}.

The fluid channel is made of an elastomeric material having a predetermined modulus of elasticity, λₑ. Structurally, the fluid channel has a first end that is connected in fluid communication with the collapsible bag. The fluid channel also defines an operational segment that extends in a distal direction from the first end toward a second end where it connects with the cannula needle set. Between its first and second ends, the fluid channel is operationally engaged with a pinch/squeeze mechanism.

Operationally, the pinch/squeeze mechanism is engaged with the elastomeric fluid channel for the dual purpose of cyclically decreasing and increasing a predetermined infusion volume, Vᵢ, in the fluid channel. In this operation, when the pinch/squeeze mechanism moves to occlude the elastomeric channel, an infusion volume Vᵢ of fluid medicament is pushed through the lumen of the channel. Also, as the channel is being occluded, the elastic material of the channel in the location of the occlusion is stressed. With this action an infusion volume Vᵢ is displaced and infused to the patient. On the other hand, when the pinch/squeeze mechanism is withdrawn to open and dilate, the elastic material of the channel becomes unstressed. It is an important aspect of the present invention that as the elastic material of the channel becomes unstressed, an infusion volume Vᵢ is drawn from the collapsible bag and into the location in the channel for subsequent engagement with the pinch/squeeze mechanism.

A controller is connected in combination with the pressure sensor, the equilibration valve and the pinch/squeeze mechanism. Specifically, the controller is used to control and coordinate the infusion of a volume Vᵢ of fluid medicament to the patient. As noted above, this operation is done primarily with the pinch/squeeze mechanism by imposing and then relieving radially directed elastomeric stresses on the fluid channel. It happens that as a volume Vᵢ is removed from the chamber, P_{c} will incrementally decrease. Thus, for the present invention it is necessary to periodically equilibrate P_{G} with the ambient pressure, P_{amb}.

Specifically, equilibration of the pressure chamber is accomplished in accordance with the predetermined pressure profile which establishes acceptable ranges and values for the decreasing changes in P_{c}. These changes are detected by the pressure sensor and monitored by the controller. Moreover, it is to be appreciated that changes in P_{c} correspond to volume changes Vᵢ of the collapsible bag within the pressure chamber.

In detail, the pressure profile establishes acceptable operating pressure ranges for P_{c} during each duty cycle Δt of the pinch/squeeze mechanism. The controller thereby monitors a pressure change ΔP_{c} for each duty cycle (ΔP_{c}/Δt). It also identifies an infusion volume Vᵢ of fluid medicament that has been infused to the patient during each duty cycle. As indicated above, the controller activates the equilibration valve to equilibrate P_{c} in the pressure chamber with the ambient pressure P_{amb} for the next duty cycle whenever there is a total pressure drop to a minimum pressure Pₘᵢₙ in the pressure chamber.

Structurally, the pinch/squeeze mechanism comprises, in combination, a base member, a piston unit, and a motorized cam shaft. In this combination, the base member is formed with an elongated U-shaped groove for receiving a portion of the fluid channel's operational segment. The groove has a first side which is formed as a platen and a second side, which is parallel to the first side, where the piston unit is located. Included with the piston unit is an upstream valve, a drive piston, and a downstream valve which are aligned in order with each other in the distal direction along the operational segment of the enclosed fluid channel positioned in the groove. The rotatable, motorized cam shaft is mounted on the base member and connected with the controller for activating the piston unit to maintain Vᵢ constant during the duty cycle in accordance with the pressure profile.

For a detailed understanding of an operation of the present invention, consider that during each duty cycle, the present invention relies on concerted work from both an elastomeric expansion of the fluid channel, Uₑ, and a sub-ambient pressurized collapse of the fluid medicament bag, Uₚ. Together, the total work, Uₜₒₜₐₗ=Uₑ+Uₚ, must always be greater than the minimum level of work, Uₘᵢₙ, that is required to move an infusion volume Vᵢ of fluid medicament from the collapsible bag and into the fluid channel (i.e., Uₜₒₜₐₗ=Uₑ+Uₚ>Uₘᵢₙ). Operationally, this concerted work must be accomplished within a predetermined time interval Δt. The import here is that, with the limitation Uₘᵢₙ in mind, the respective forces for doing the work Uₑ and Uₚ will predictably diminish with time.

In this context, an infusion volume, Vᵢ, must be infused to a patient during the predetermined time interval Δt. Thus, the infusion rate Vᵢ/Δt, must be maintained to ensure that Uₑ+Uₚ>Uₘᵢₙ. Stated differently, the infusion rate Vᵢ/Δt needs to be continuously satisfied by the combined effects of an elastomeric expansion of the fluid channel, and the collapse of the fluid bag in the pressurized chamber. An operational analysis of this relationship is best appreciated by separate considerations of Uₑ and Uₚ.

From a materials perspective, work done by the expanding elastomeric channel is a function of the modulus of elasticity λₑ of the elastomeric material that is used to manufacture the channel. In the event, as the elastomeric material rebounds from a stressed condition, to thereby open the fluid channel at the location where it was squeezed, it will do the work Uₑ. By analogy, Uₑ can be considered as the action of a radial force, Fᵣ, acting through a distance d, in a direction perpendicular to fluid flow, at the location on the elastomeric fluid channel where it was squeezed. As noted above, Fᵣ is generated by internal forces dependent on λₑ of the material.

The overall consequence from the diminishing values of Fᵣ and P_{c} is that both Uₑ and Uₚ diminish over time, albeit at different rates. In the case of Uₚ, however, P_{c} can be periodically equilibrated to P_{amb} on a short-term basis. In contrast, Uₑ has no such short-term reenergizing capability. Nevertheless, an efficient operation is possible as long as Uₜₒₜₐₗ=Uₑ+Uₚ>Uₘᵢₙ is satisfied and Vᵢ/Δt can be maintained.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an interactive layout of operative components for the present invention and their cooperative interactions;

FIG. 2 is a pressure profile in accordance with the present invention;

FIGS. 3A-D show sequential configurations of the piston unit of a pinch/squeeze mechanism during a duty cycle in accordance with the present invention, wherein FIG. 3A is a pre-fill configuration, FIG. 3B is a fill configuration, FIG. 3C is a pre-dispense configuration, and FIG. 3D is a dispense configuration; and

FIG. 4 is a logic flow chart for the controller operation implementing the pressure profile.

### DESCRIPTION OF THE PREFERRED EMBODIMENTS

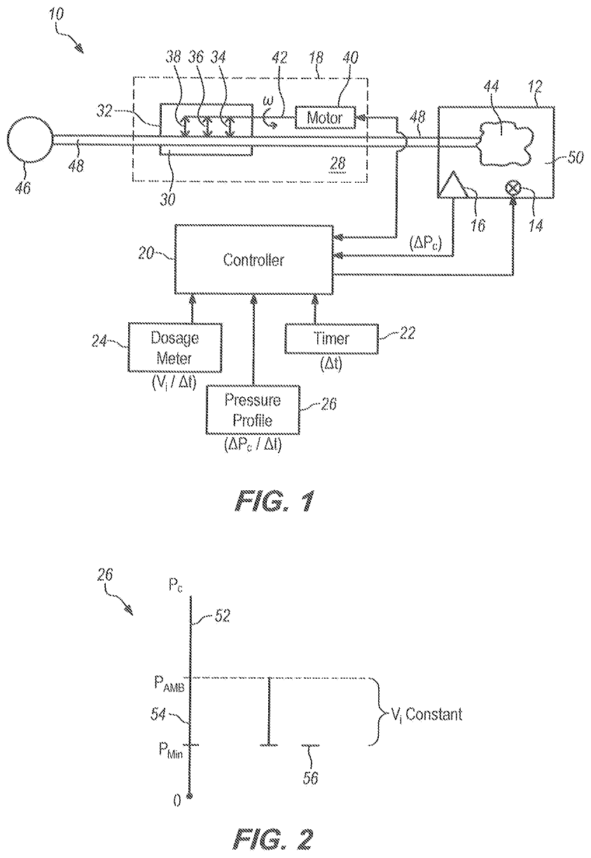

Referring initially to FIG. 1, a schematic diagram of an infusion pump in accordance with the present invention is shown and generally designated 10. As shown, the pump 10 includes a pressure shell 12 on which is mounted an equilibration valve 14 and a pressure sensor 16. Also included with the pump 10 is a pinch/squeeze mechanism 18 and a controller 20. In detail, the controller 20 is connected with the equilibration valve 14 and a pressure sensor 16 on the pressure shell 12. An airtight pressure chamber 50 that is selectively enclosed within the pressure shell 12. The controller 20 is also electronically connected with the pinch/squeeze mechanism 18.

FIG. 1 further shows that the controller 20 includes a timer 22 for monitoring, establishing, and controlling a duty cycle Δt for the pump 10. The controller 20 also includes a dosage meter 24 which is set by a user/patient (not shown) to establish the appropriate infusion volume Vᵢ that is be infused to the user during each duty cycle (Vᵢ/Δt). A pressure profile 26, is included with the controller 20 which provides detailed instructions insofar as an operation of the pump 10 is concerned for compliance with a clinically provided, predetermined operational protocol.

Still referring to FIG. 1, the pinch/squeeze mechanism 18 is shown to include a base member 28 having a platen 30 and an opposed piston unit 32 mounted thereon. In detail, the piston unit 32 includes an upstream valve 34, a drive piston 36, and a downstream valve 38 which are aligned sequentially with each other on the piston unit 32, and across from the platen 30, to form a groove on the base member 28 therebetween. Also mounted on the base member 28 is a motor 40 for rotating a cam shaft 42. In combination, the cam shaft 42 is engaged with the piston unit 32, and the motor 40 is connected with the controller 20 for rotating the cam shaft 42 at an appropriate angular velocity ω. As intended for the present invention, the angular velocity ω is established by the controller 20 to establish the desired infusion volume Vᵢ of fluid medicament during each duty cycle Δt.

As intended for the present invention, the pump 10 is designed for an operational engagement with a disposable infusion unit that includes: a collapsible bag 44 for holding the fluid medicament that is to be infused to the patient, a cannula needle set 46, and an elastomeric fluid channel 48 that is connected to establish fluid communication between the collapsible bag 44 and the cannula needle set 46. Structurally, the collapsible bag 44 is dimensioned to be received into a pressure chamber 50 that is created when the pressure shell 12 is closed.

Several important structural/functional characteristics of the pressure chamber 50 must be satisfied when the pressure chamber 50 is closed. For one, the pressure chamber 50 must be airtight when the pressure shell 12 is closed. For another, the air volume of the pressure chamber 50 inside a closed pressure shell 12 must be greater than the volume of the collapsible bag 44, when the collapsible bag 44 is filled to its full capacity. This is done to optimize the efficacy of an external sub-ambient pressure in the pressure chamber 50 against the collapsible bag 44 during an operation of the pump 10. Further, the chamber pressure P_{c}, as measured by the pressure sensor 16 inside the pressure chamber 50, must be continuously monitored by the controller 20 during an operation of the pump 10.

With consideration of the elastomeric fluid channel 48, it is important that the fluid channel 48 be made of an elastomeric material which has a modulus of elasticity λₑ that causes a relatively rapid transition (rebound/reset) from a stressed configuration back to an unstressed configuration. Specifically, the elastomeric fluid channel 48 is structurally formed with a lumen to transport fluid medicament from the collapsible bag 44 to the cannula needle set 46. In an operation of the present invention, this requires that a portion of the fluid channel 48 be cyclically stressed (collapsed) and unstressed (dilated) by a radially acting, reciprocating force ±F as the lumen of the fluid channel 48 is mechanically collapsed (+F) by the piston unit 32 and dilated by elastomeric forces (−F) from the fluid channel 48. From an operational perspective, this action causes the elastomeric material of the fluid channel 48 to generate a force (−F) that reopens the lumen of the fluid channel 48, and assists the force P_{c} in the pressure chamber 50 in drawing fluid medicament from the collapsible bag 44 and into the elastomeric fluid channel 48.

A pressure profile in accordance with the present invention is shown in FIG. 2, where it is generally designated 26. In FIG. 2 it is shown that the pressure profile 26 includes a line graph 52 of pressures P_{c} for the pressure chamber 50. Specifically, line graph 52 indicates that the pressure profile 26 has an operating range 54 that extends between an ambient pressure P_{amb} and a minimum pressure Pₘᵢₙ. Importantly, FIG. 2 shows the relationship between the operating range 54 of the pump 10 and the ambient pressure P_{amb} of the environment where the pump 10 is to be operated. Moreover, FIG. 2 indicates that P_{c} is to be monitored and controlled by the controller 20 to maintain a constant infusion volume Vᵢ for the patient during an operation of the pump 10. FIG. 2 also indicates the pressure profile 26 establishes an equilibration point 56, at the pressure Pₘᵢₙ), where P_{c} is equilibrated back to P_{amb} for a continued operation of the pump 10.

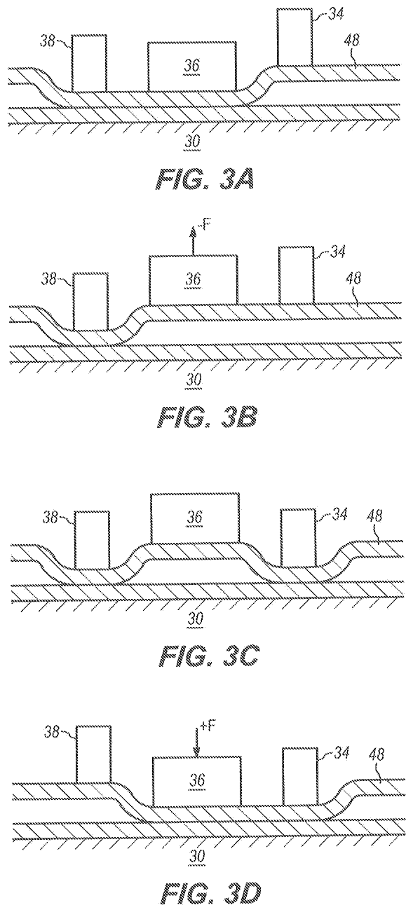

With reference to FIGS. 3A-3D, it is to be appreciated that the pressure profile 26 is based on a duty cycle for the piston unit 32 having a time duration, Δt. Functionally, Δt is the time duration for each sequential 360° rotation of the cam shaft 42. Further, during each Δt, the radially directed interactive forces ±F of the drive piston 36 and the fluid channel 48, respectively, are accomplished during each duty cycle tit by a sequence of configurations for the piston unit 32.

In FIG. 3A, a pre-fill configuration is shown for the piston unit 32 wherein the downstream valve 38 is closed, the drive piston 36 has been radially advanced to stress the fluid channel 48 against the platen 30, and the upstream valve 34 is open to establish fluid communication between the piston unit 32 and fluid medicament in the collapsible bag 44.

FIG. 3B shows a fill configuration for the piston unit 32 wherein the downstream valve 38 remains closed, while the upstream valve 34 remains open, and the drive piston 36 is radially withdrawn from the platen 30 to unstress the enclosed fluid channel 48 for an elastic rebound with a force −F from its stressed configuration. Thus, fluid medicament is drawn from the collapsible bag 44 by elastomeric rebound of the fluid channel 48 and into the fluid channel 48 as the fluid channel 48 dilates during rebound from its stressed configuration.

FIG. 3C shows a pre-dispense configuration for the piston unit 32 wherein the downstream valve 38 remains closed, the drive piston 36 remains withdrawn from the platen 30 and the upstream valve 34 is closed.

Finally, in FIG. 3D, a dispense configuration for the piston unit 32 is shown wherein the upstream valve 34 remains closed, the downstream valve 38 is opened, and the drive piston 36 is radially advanced with a force +F toward the platen 30 to pump fluid medicament from the piston unit 32 in a distal direction downstream into the operational segment of the fluid channel 48 for infusion to the patient.

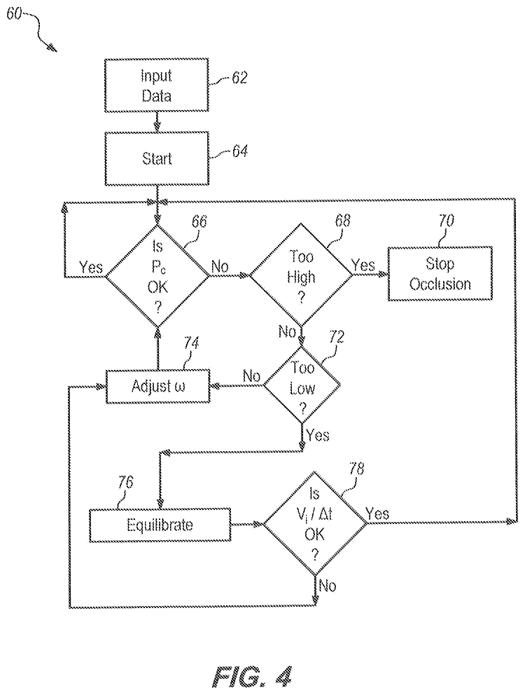

An operation of the pump 10, in accordance with the pressure profile 26, will be best appreciated with reference to the logic flow chart 60 shown in FIG. 4. There it will be seen that the action block 62 requires input data. Specifically, this input data will include the value for Pₘᵢₙ required for the pressure profile 26. The input data will also require values for the fluid medicament infusion volume Vᵢ and a start value for the duty cycle Δt. With required input, action block 64 indicates that pump 10 can be started.

At the start of an operation of the pump 10, inquiry block 66 determines whether the chamber pressure P_{c} in the pressure chamber 50 is OK. According to inquiry block 66, if the answer is YES, the operation continues. However, if the answer is NO, inquiry block 68 determines whether P_{c} is above P_{amb}. From this inquiry, if P_{c}>P_{amb} an occlusion may be indicated and, in accordance with action block 70, the pump 10 should be stopped.

On the other hand, if P_{c}<P_{amb}, inquiry block 72 determines whether P_{c} is too low. Stated differently, the inquiry block 72 determines whether P_{c} is within the operating range 54 established by the pressure profile 26 (see FIG. 2). If the response from inquiry block 72 is NO, indicating that P_{c} is still within the operating range 54, the action block 74 indicates that, for continued operation, an optional action is to adjust the angular velocity ω of cam shaft 42. It is noted that adjusting w will also result in a change of the duty cycle Δt for pump 10 which, for any number of reasons, may be desirable.

When the response of inquiry block 72 is YES, the action block 76 indicates that the controller 20 will activate the equilibration valve 14 on pressure shell 12. This is done to equilibrate P_{c} in the pressure chamber 50 of pressure shell 12 with the ambient pressure P_{amb}. The next determination for the operation of the pump 10 is indicated by inquiry block 78, where Vᵢ is evaluated in the context of the duty cycle Δt. Specifically, this evaluation begins with P_{c}=P_{amb} when the response of inquiry block 78 is YES, and it continues through subsequent successive duty cycles Δt for as long as inquiry block 72 indicates the pressure profile 26 is satisfied. Thus, it is inquiry block 78 that determines when P_{c} requires equilibration. FIG. 4 also shows that when the response of inquiry block 78 is NO, it may be necessary to adjust ω of motor 40.

While the particular Portable Infusion Pump with Negative Pressure Control as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A portable pump for infusing a fluid medicament to a patient which comprises:
a pressure shell adapted for enclosing an accessible pressure chamber with an airtight seal, wherein the pressure chamber has a predetermined volume V_{c};
a pressure sensor mounted on the pressure shell to monitor a chamber pressure P_{c} within the pressure chamber of the pressure shell;
an equilibration valve mounted on the pressure shell to equilibrate the chamber pressure P_{c} with the ambient pressure;
a collapsible bag for holding a volume of the fluid medicament less than V_{c};
an enclosed fluid channel made of an elastomeric material having a predetermined modulus of elasticity λₑ, wherein the fluid channel has a first end connected in fluid communication with the collapsible bag, and an operational segment extending in a distal direction from the first end toward a second end of the fluid channel, with a lumen formed in the fluid channel between the first and second ends;
a pinch/squeeze mechanism engaged with a portion of the operational segment of the elastomeric fluid channel for decreasing and increasing a predetermined infusion volume Vᵢ in the fluid channel during a duty cycle Δt, wherein Vᵢ is the decrease/increase change difference in fluid volume within the elastomeric fluid channel along a predetermined length of the elastomeric fluid channel; and
a controller connected with the pressure sensor, the equilibration valve and the pinch/squeeze mechanism, for sequentially operating the pinch/squeeze mechanism to infuse a volume Vᵢ of fluid medicament to the patient by imposing elastomeric stress on the fluid channel to draw a volume Vᵢ of fluid medicament from the collapsible bag in response to both an elastomeric reset of the fluid channel and pressure P_{c} on the collapsible bag in the pressure chamber, and to periodically equilibrate P_{c} with the ambient pressure P_{amb} in accordance with a pressure profile responsive to decreasing changes in P_{c} detected by the pressure sensor, corresponding to volume changes Vᵢ of the collapsible bag within the pressure chamber.

2. The infusion pump of claim 1 wherein the pressure profile establishes acceptable operating pressure ranges for P_{c} during a duty cycle Δt for controller-monitoring a pressure change ΔP_{c} for each duty cycle (ΔP_{c}/Δt), and identifies an infusion volume Vᵢ of fluid medicament for infusion to the patient during each duty cycle, and further wherein the controller activates the equilibration valve to equilibrate P_{c} in the pressure chamber with the ambient pressure P_{amb} for the next duty cycle whenever there is a total pressure drop to a minimum pressure Pₘᵢₙ in the pressure chamber.

3. The infusion pump of claim 2 wherein the pinch/squeeze mechanism comprises:
a base member formed with an elongated U-shaped groove for receiving a portion of the fluid channel's operational segment therein, wherein the groove has a first side formed as a platen and a second side parallel to the first side and located across the groove therefrom to establish the groove therebetween;
a piston unit supported on the second side of the groove, wherein the piston unit includes an upstream valve, a drive piston, and a downstream valve aligned in order with each other in the distal direction along the operational segment of the enclosed fluid channel positioned in the groove; and
a rotatable, motorized cam shaft mounted on the base member and connected with the controller for activating the piston unit to maintain Vᵢ constant during the duty cycle in accordance with the pressure profile.

4. The infusion pump of claim 3 wherein the pressure profile establishes a duty cycle for the piston unit having a time duration, Δt, for each sequential 360° rotation of the cam shaft, and wherein a radially directed interaction of the drive piston with the portion of the enclosed fluid channel axially oriented in the groove is accomplished during each duty cycle by a sequence of configurations comprising:
a pre-fill configuration wherein the downstream valve is closed, the drive piston is radially advanced to collapse and stress the fluid channel against the platen, and the upstream valve is open to establish fluid communication between the piston unit and fluid medicament in the collapsible bag;
a fill configuration wherein the downstream valve remains closed, while the upstream valve remains open, and the drive piston is radially withdrawn from the platen to unstress the enclosed fluid channel for an elastic rebound from its stressed configuration, to thereby draw fluid medicament from the collapsible bag and into the fluid channel in the groove as the fluid channel dilates during rebound from its stressed configuration;
a pre-dispense configuration wherein the downstream valve remains closed, the drive piston remains withdrawn from the platen and the upstream valve is closed; and
a dispense configuration wherein the upstream valve remains closed, the downstream valve is opened, and the drive piston is radially advanced toward the platen to pump fluid medicament from the piston unit in a distal direction downstream into the operational segment of the fluid channel for infusion to the patient.

5. The infusion pump of claim 2 wherein an occlusion is indicated when an aberration occurs at a Pₘₐₓ in the pressure profile.

6. The infusion pump of claim 4 wherein dosage corrections required for compliance with the pressure profile are accomplished by adjusting the rotation rate ω of the cam shaft by ±ω within a predetermined range.

7. The infusion pump of claim 4 wherein Vᵢ is determined at a location where the piston unit interacts with the elastomeric fluid channel through a distance d to collapse the elastomeric channel and displace a volume Vᵢ in the lumen of the elastomeric fluid channel.

8. The infusion pump of claim 4 wherein Vᵢ is determined empirically.

9. A portable infusion pump for infusing a fluid medicament to a patient which comprises:
an infusion unit including a collapsible bag for holding the fluid medicament, a cannula needle set, and an elastomeric fluid channel interconnecting the collapsible bag in fluid communication with the cannula needle set;
a means for holding the collapsible bag in an airtight pressure chamber;
a means for manipulating the elastomeric fluid channel to draw fluid medicament from the collapsible bag and thereafter transfer fluid medicament to the cannula needle set;
an equilibrator connected to the holding means;
a pressure sensor for monitoring a pressure P_{c} in the pressure chamber, and
a controller connected to the pressure chamber, the equilibrator, the pressure sensor, and to the manipulating means for operating the manipulating means to draw fluid medicament from the collapsible bag and to transfer fluid medicament to the cannula needle set in accordance with a predetermined pressure profile for the pressure chamber.

10. The infusion pump of claim 9 wherein the holding means comprises a pressure shell adapted for enclosing the pressure chamber with a predetermined volume V_{c}, and wherein the equilibrator comprises an equilibration valve mounted on the pressure shell and connected with the controller to periodically equilibrate P_{c} with the ambient pressure P_{amb}.

11. The infusion pump of claim 10 wherein the elastomeric fluid channel has a predetermined modulus of elasticity λₑ, and wherein the fluid channel has a first end connected in fluid communication with the collapsible bag and an operational segment extending in a distal direction from the first end toward a second end of the fluid channel, with a lumen formed in the fluid channel between the first and second ends.

12. The infusion pump of claim 11 wherein the manipulating means comprises:
a pinch/squeeze mechanism engaged with a portion of the operational segment of the elastomeric fluid channel for periodically decreasing and increasing a predetermined infusion volume Vᵢ in the lumen of the fluid channel during a duty cycle Δt, wherein Vᵢ is the decrease/increase change difference in fluid volume within the elastomeric fluid channel along a predetermined length of the elastomeric fluid channel; and
a controller connected with the pressure sensor, the equilibration valve and the pinch/squeeze mechanism, for sequentially operating the pinch/squeeze mechanism to infuse a volume Vᵢ of fluid medicament to the patient by imposing an elastomeric stress on the fluid channel, and thereafter relieving the elastomeric stress on the fluid channel to draw a volume Vᵢ of fluid medicament from the collapsible bag, along with a commensurate decrease in pressure P_{c} on the bag in the pressure chamber, and to periodically equilibrate P_{c} with the ambient pressure P_{amb}, when P_{c} equals Pₘᵢₙ, in accordance with the predetermined pressure profile.

13. The infusion pump of claim 12 wherein the pinch/squeeze mechanism comprises:
a base member formed with an elongated U-shaped groove for receiving a portion of the fluid channel's operational segment therein,
wherein the groove has a first side formed as a platen and a second side parallel to the first side and located across the groove therefrom to establish the groove therebetween;
a piston unit supported on the second side of the groove, wherein the piston unit includes an upstream valve, a drive piston, and a downstream valve aligned in order with each other in the distal direction along the operational segment of the enclosed fluid channel positioned in the groove; and
a rotatable, motorized cam shaft mounted on the base member and connected with the controller for activating the piston unit in accordance with the pressure profile.

14. The infusion pump of claim 13 wherein the pressure profile establishes a duty cycle for the piston unit having a time duration, Δt, for each sequential 360° rotation of the cam shaft, and wherein a radially directed interaction of the drive piston with the portion of the enclosed fluid channel axially oriented in the groove is accomplished during each duty cycle by a sequence of configurations comprising:
a pre-fill configuration wherein the downstream valve is closed, the drive piston is radially advanced to occlude and stress the fluid channel against the platen, and the upstream valve is open to establish fluid communication between the piston unit and fluid medicament in the collapsible bag;
a fill configuration wherein the downstream valve remains closed, while the upstream valve remains open, and the drive piston is radially withdrawn from the platen to unstress the enclosed fluid channel for an elastic rebound from its stressed configuration, to thereby draw fluid medicament from the collapsible bag and into the fluid channel in the groove as the fluid channel dilates during rebound from its stressed configuration;
a pre-dispense configuration wherein the downstream valve remains closed, the drive piston remains withdrawn from the platen and the upstream valve is closed; and
a dispense configuration wherein the upstream valve remains closed, the downstream valve is opened, and the drive piston is radially advanced toward the platen to pump fluid medicament from the piston unit in a distal direction downstream into the operational segment of the fluid channel for infusion to the patient.

15. A method for infusing a fluid medicament to a patient through an elastomeric fluid channel during a duty cycle Δt which comprises the steps of:
providing an infusion unit wherein an elastomeric fluid channel extends between a first end and a second end with a collapsible bag filled with fluid medicament attached to the first end and a cannula needle set attached to the second end;
using a mechanical means engaged with the elastomeric fluid channel at a predetermined location to impose a radially directed mechanical force +F against the elastomeric fluid channel to stress the fluid channel and move a first infusion volume Vᵢ of fluid medicament from the mechanical means to the cannula needle set, for infusion to the patient during a first part of Δt; and
maintaining a sub-ambient pressure P_{amb} on the collapsible bag to cooperate with an elastomeric force −F generated as the elastomeric fluid channel is unstressed during a second part of Δt, to draw a second infusion volume Vᵢ of fluid medicament from the collapsible bag to the location of the mechanical means for a next duty cycle Δt.

16. The method of claim 15 further comprising the steps of:
positioning the collapsible bag in a pressure chamber of an airtight pressure shell;
monitoring a chamber pressure P_{c} inside the pressure chamber to determine changes ΔP_{c}/Δt during each duty cycle; and
evaluating ΔP_{c}/Δt for compliance with a predetermined pressure profile.

17. The method of claim 16 wherein the pressure profile establishes values for operational chamber pressures of P_{c} between Pₘᵢₙ and ambient pressure P_{amb}.

18. The method of claim 17 further comprising the step of equilibrating the pressure chamber to P_{amb} when P_{c} equals Pₘᵢₙ.

19. The method of claim 18 further comprising the step of ceasing the using step when P_{c} is greater than Pₘₐₓ.
